(19) United States
(12) Patent Application Publication
(10) Pub. No.: US 2025/0295447 A1
(43) Pub. Date: Sep. 25, 2025

(54) HANDLE ASSEMBLY AND ENDOSCOPE TREATMENT TOOL

(71) Applicant: OLYMPUS MEDICAL SYSTEMS CORP., Tokyo (JP)

(72) Inventors: Kotaro YAMADA, Hachioji-shi (JP); Kikuo TAKI, Hino-shi (JP); Daiki HAGIWARA, Kokubunji-shi (JP); Kentaro KONDO, Hachioji-shi (JP)

(21) Appl. No.: 19/040,512

(22) Filed: Jan. 29, 2025

Related U.S. Application Data
(60) Provisional application No. 63/569,475, filed on Mar. 25, 2024.

(51) Int. Cl. A61B 18/14; A61B 1/005; A61B 1/018

(52) U.S. Cl. CPC A61B 18/1482; A61B 1/005; A61B 1/018; A61B 18/1445

(57) ABSTRACT

A handle assembly includes: a handle body; a transmission member configured to transmit a high-frequency current; a connector being conductive and extending in a direction intersecting a longitudinal axis of the transmission member; and an elastic body being conductive, wherein the elastic body contacts the transmission member in a state in which the elastic body is compressed, and the transmission member is slidable relative to the connector in a state in which the transmission member is pressed against the connector.

### CROSS-REFERENCE TO RELATED APPLICATIONS

The present application claims priority based on U.S. Patent Provisional Application No. 63/569,475 filed in the United States on Mar. 25, 2024, the contents of which are incorporated herein by reference.

### TECHNICAL FIELD

This disclosure relates to an endoscope treatment tool.

### BACKGROUND

Conventionally, in endoscope treatment, an endoscope treatment tool is used that includes a high-frequency treatment device that applies high-frequency current, such as a hemostatic forceps that cauterize a bleeding treatment target to stop the bleeding, a high-frequency knife, etc. A high-frequency current is supplied to a long member such as a wire connected to the high-frequency treatment device via a power cable connected to a connector provided in an operating portion of the endoscope treatment tool (for example, Japanese Unexamined Patent Application, First Publication No. 2008-295905 and No. 2009-034388).

However, in an endoscope treatment tool equipped with a high-frequency treatment device, unless the electrical connection between the connector to which high-frequency current is supplied and a long member such as a wire connected to the high-frequency treatment device is reliably secured, the high-frequency treatment device will not be able to exhibit good treatment capabilities.

### SUMMARY

The present disclosure provides a handle assembly and an endoscope treatment tool that reliably ensure electrical connection between a connector to which high-frequency current is supplied and an elongated member such as a wire connected to a high-frequency treatment device.

A handle assembly of an embodiment includes: a handle body; a transmission member configured to transmit a high-frequency current; a connector being conductivity and extending in a direction intersecting a longitudinal axis of the transmission member; and an elastic body being conductivity, wherein the elastic body contacts the transmission member in a state in which the elastic body is compressed, and the transmission member is slidable relative to the connector in a state in which the transmission member is pressed against the connector.

The endoscope treatment tool according to the present disclosure reliably ensures electrical connection between the connector to which high-frequency current is supplied and an elongated member such as a wire connected to a high-frequency treatment device.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an overall view of an endoscope treatment system according to a first embodiment.

FIG. 2 is an overall view showing an endoscope treatment tool of the endoscope treatment system.

FIG. 3 is a cross-sectional view of an operating portion of the endoscope treatment tool.

FIG. 4 is a cross-sectional view of a slider of the operating portion.

FIG. 5 is a view showing a coil spring of the operating portion.

FIG. 6 is a cross-sectional view of the coil spring.

FIG. 7 is a view showing an elastic spring which is a modification of the coil spring.

FIG. 8 is a view showing another aspect of the coil spring.

FIG. 9 is a view showing another aspect of the coil spring.

FIG. 10 is a cross-sectional view of an operating portion of an endoscope treatment tool according to a second embodiment.

FIG. 11 is a view showing a leaf spring which is a modification of the coil spring of the operating portion.

FIG. 12 is a view showing the leaf spring.

FIG. 13 is a view showing the leaf spring.

FIG. 14 is a cross-sectional view of an operating portion of an endoscope treatment tool according to a third embodiment.

FIG. 15 is a cross-sectional view of an operating portion of an endoscope treatment tool according to a fourth embodiment.

FIG. 16 is a perspective view of the operating portion.

FIG. 17 is a diagram showing a current path from an electrical connector to a pipe.

FIG. 18 is a diagram showing a modification of the above-mentioned operating portion.

FIG. 19 is a diagram showing the treatment portion (end effector).

### DETAILED DESCRIPTION

### First Embodiment

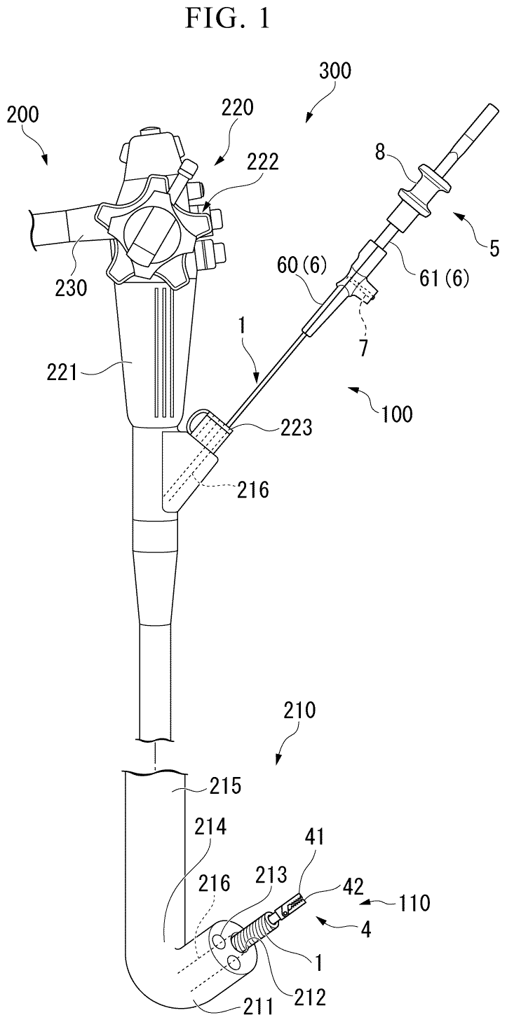

An endoscope treatment system 300 including an endoscope treatment tool 100 according to a first embodiment of the present disclosure will be described with reference to FIGS. 1 to 9. FIG. 1 is an overall view of the endoscope treatment system 300.

### [Endoscope Treatment System 300]

As shown in FIG. 1, the endoscope treatment system 300 includes an endoscope treatment tool 100 and an endoscope 200. The endoscope treatment tool 100 is inserted into the endoscope 200 for use.

### [Endoscope 200]

The endoscope 200 is a known flexible endoscope, and includes an insertion portion 210 that is inserted into the body from the distal end, an operating portion 220 attached to the proximal end of the insertion portion 210, and a universal cord 230 attached to the operating portion 220.

The insertion portion 210 is a long, thin member that can be inserted into a lumen. The insertion portion 210 has a distal end portion 211, a bending portion 214, and a flexible portion 215. The distal end portion 211, the bending portion 214, and the flexible portion 215 are connected in this order from the distal side. Inside the insertion portion 210, a channel 216 that inserts the endoscope treatment tool 100 is provided.

The distal end portion 211 is provided with a distal end opening 212 of the channel 216 and an imaging portion 213.

The imaging portion 213 is equipped with an imaging element such as a CCD or CMOS, and is capable of imaging the area to be treated. The bending portion 214 bends according to the user's operation of the operating portion 220. The flexible portion 215 is a flexible tubular portion.

The operating portion 220 is connected to the flexible portion 215. The operating portion 220 has a grip 221, an input portion 222, and a forceps port 223. The grip 221 is a member supported by the user. The input portion 222 accepts an operation input for bending the bending portion 214. The forceps port 223 is the proximal end opening of the channel 216.

The universal cord 230 connects the endoscope treatment tool 100 to an external device. An imaging cable or an optical fiber cable that outputs an image signal captured by the imaging portion 213 to the outside is inserted into the universal cord 230.

### [Endoscope Treatment Tool 100]

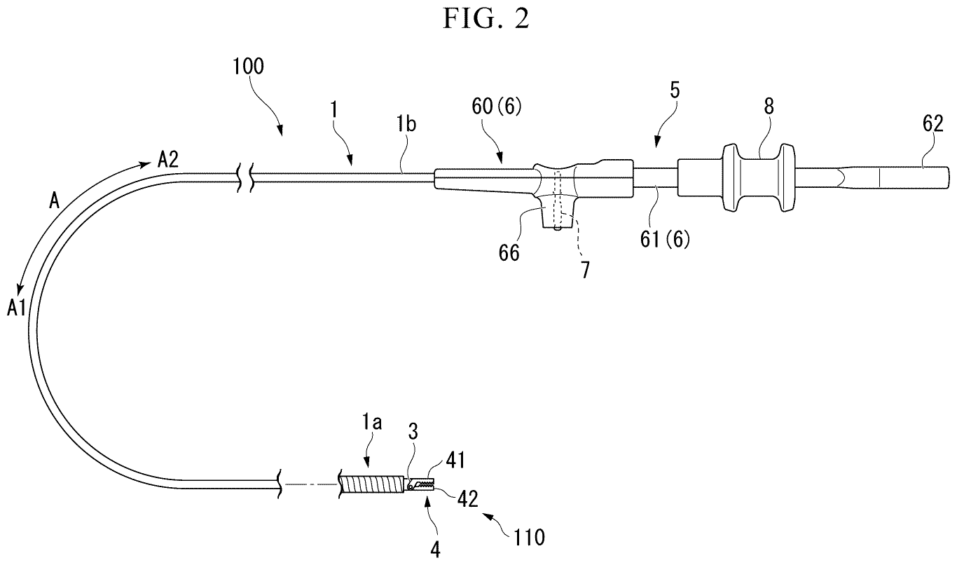

FIG. 2 is an overall view of the endoscope treatment tool 100.

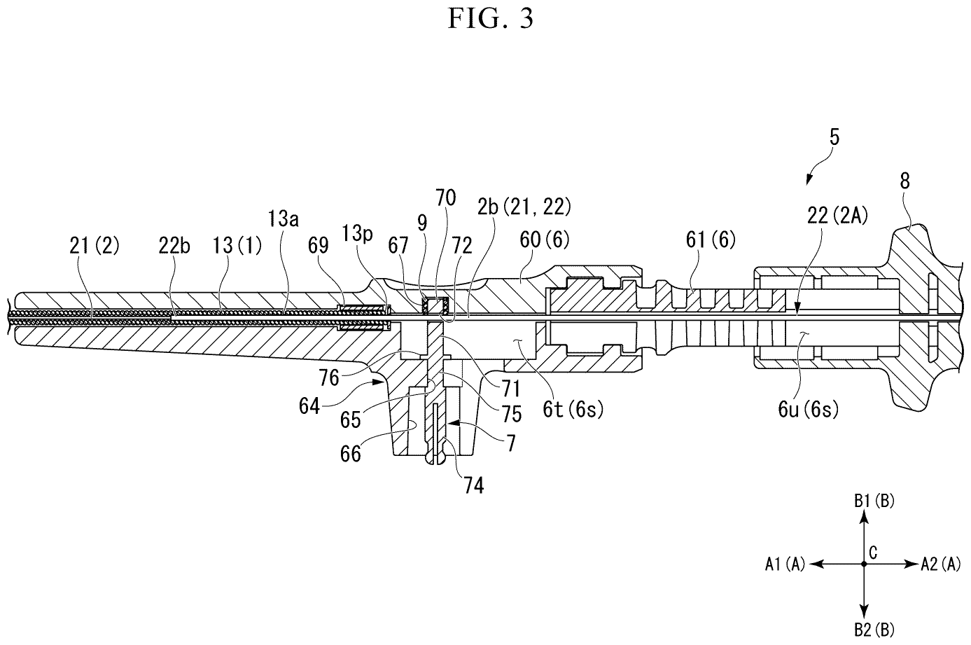

The endoscope treatment tool 100 (also called the treatment tool 100) is a hemostatic forceps that cauterizes the affected area to stop bleeding. The treatment tool 100 includes a sheath 1, a long member 2 (see FIG. 3), a transmission member 2A (see FIG. 3), a support member 3, forceps (jaw) 4, and an operating portion 5. In the following description, in the longitudinal axis direction A of the treatment tool 100, the side inserted into the patient's body is referred to as the “distal end side (distal side) A1” and the operating portion 5 side is referred to as the “proximal end side (proximal side) A2”.

The sheath 1 has a flexible, long coil sheath 13 extending from the distal end 1a to the proximal end 1b. The sheath 1 has an outer diameter that allows it to be inserted into the channel 216 of the endoscope 200. The sheath 1 may have insulating properties, and for example, the coil sheath 13 may be covered with a resin heat shrink tube. As shown in FIG. 1, when the sheath 1 is inserted into the channel 216, the distal end 1a of the sheath 1 can protrude and retract from the distal end opening 212 of the channel 216. The proximal end 1b of the sheath 1 is connected to the operating portion 5.

FIG. 3 is a cross-sectional view of the operating portion 5. The long member 2 passes through the internal space of the coil sheath 13. The distal end of the long member 2 is connected to the forceps 4, and the proximal end of the long member 2 is connected to the operating portion 5. The long member 2 is conductive and transmits high-frequency current to the forceps 4. The long member 2 may be a metal wire 21. The long member 2 is not limited to the wire 21, and may be any conductive member such as a metal coil tube.

The transmission member (shaft) 2A passes through the internal space 6s of the handle body 6. The transmission member 2A may be a pipe 22. The transmission member 2A is not limited to the pipe 22, as long as it is at least partially a conductive member and has higher rigidity than the wire 21.

The distal end of the wire 21 is connected to the forceps 4 via the support member 3. The proximal end of the wire 21 is inserted into the pipe (transmission member) 22 in the handle body 6. The wire 21 and the pipe 22 are fixed so that they cannot move relative to each other by, for example, a chemical bond such as adhesion or a mechanical bond such as crimping. The wire 21 is inserted through the coil sheath 13, and the distal end of the pipe 22 is inserted into the proximal end 13a of the coil sheath 13.

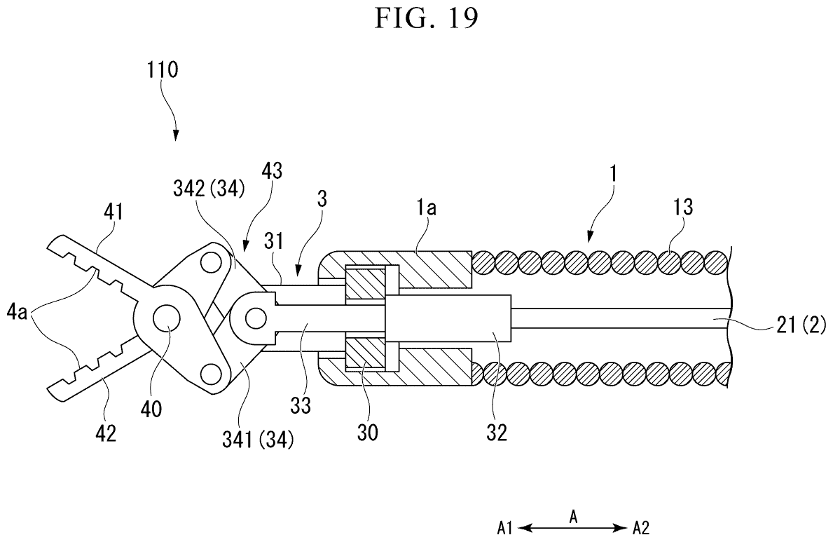

The support member 3 is provided at the distal end 1a of the sheath 1, and supports the forceps 4 so that they can be opened and closed. The support member 3 has a link mechanism 34 (see FIG. 19) that converts the advancing and retracting movement of the wire 21 into the opening and closing movement of the forceps 4.

The forceps (jaw) 4 are a member that grasps biological tissue. The forceps 4 are supported by the support member 3 so that they can be opened and closed toward the distal side A1. The forceps 4 are formed of a metal material such as stainless steel, and includes a first forceps piece 41 and a second forceps piece 42. The support member 3 and the forceps 4 constitute a “treatment portion (end effector) 110” that treats the affected area. The forceps 4 are an example of a high-frequency treatment device.

As shown in FIG. 2, the operating portion (handle, handle assembly) 5 is provided on the proximal end side A2 of the sheath 1. The operating portion 5 includes a handle body 6, an electric connector 7, a slider 8, and a coil spring 9. In this embodiment, the electric connector 7 is attached to the handle body 6.

In the following description of the operating portion 5, the direction in which the electric connector 7 is provided with respect to the handle body 6 is referred to as the lower side B2 in the vertical direction B, and the opposite side of the lower side B2 in the vertical direction B is referred to as the “upper side B1.” In addition, the direction perpendicular to the longitudinal axis direction A and the vertical direction B is referred to as the “width direction C” or the “left-right direction C.” When looking from the distal side Al to the proximal end side A2, the direction facing right is referred to as the “right side C1” in the width direction C, and the direction facing left is referred to as the “left side C2” in the width direction C.

As shown in FIG. 3, the handle body 6 has an internal space 6s through which the wire 21 can be inserted. The wire 21 passes through the internal space of the sheath 1 and the internal space 6s of the handle body 6, and extends to the slider 8. The handle body 6 has a thumb ring 62, a connector support portion 64, and a coil sheath support portion 69.

The handle body 6 has a first handle body 60 and a second handle body 61. The second handle body 61 is connected to the proximal end of the first handle body 60. The first handle body 60 and the second handle body 61 are connected so as to be relatively rotatable about a rotation axis extending in the longitudinal axis direction A. The internal space 6s is a continuous space formed in the first handle body 60 and the second handle body 61. The internal space 6s has an internal space 6t formed in the first handle body 60 and an internal space 6u formed in the second handle body 61.

The thumb ring 62 is provided on the proximal end side A2 of the second handle body 61. The surgeon can support the second handle body 61 by inserting his thumb through the thumb ring 62.

As shown in FIG. 3, the connector support portion 64 is provided on the first handle body 60. The connector support portion 64 supports the electric connector 7. The connector support portion 64 has a through hole 65 penetrating in the vertical direction B, a cylindrical plug socket 66 formed on the lower side B2 of the through hole 65, and a coil spring support portion 67.

As shown in FIG. 3, the electric connector 7 is supported by the connector support portion 64, is a conductive member extending in a direction intersecting the longitudinal axis of the pipe 22, and is formed in an approximately cylindrical shape. Note that “approximately cylindrical” includes a shape close to a cylindrical shape in addition to a strict cylindrical shape. In this embodiment, the electric connector 7 extends in the vertical direction B.

The electrical connector 7 has an upper end 70, a connecting portion 71, an energized plug 74, a reduced diameter portion 75, and a flange 76. The electrical connector 7 extends between the energized plug (first end) 74 and an upper end (second end) 70 supported by the coil spring support portion 67. The connecting portion 71 and the reduced diameter portion 75 are located between the energized plug 74 and the upper end 70. The upper end 70 is provided on the upper side B1 of the connecting portion 71. The extension direction of the electrical connector 7 and the extension direction of the pipe 22 (wire 21) intersect.

The connecting portion 71 is formed in a substantially cylindrical shape, and is provided on the upper side B1 of the electrical connector 7. The connecting portion 71 is connected to the pipe 22 in the internal space 6t of the first handle body 60. An insertion passage 72 is formed in the connecting portion 71. That is, an insertion passage 72 extending in the longitudinal direction of the first handle body 60 is formed at the proximal end of the electrical connector 7.

The pipe 22 is inserted into the passage 72. That is, the passage 72 is a through hole formed along the longitudinal axis direction A in which the pipe 22 extends, and the pipe 22 and the wire 21 passing through the pipe 22 are inserted so as to be able to advance and retract. In the following description, the wire 21 and the pipe 22 passing through the passage 72 are also referred to as the “proximal end connection portion 2b”.

The electric plug 74 is a plug provided on the lower side B2 (first end of the electric connector 7) of the electric connector 7, and a power cable (active cord) is connected to it.

The reduced diameter portion 75 is an intermediate part in the vertical direction B, and is provided between the passage 72 and the electric plug 74. The outer diameter of the reduced diameter portion 75 is smaller than the outer diameter of the connecting portion 71 and the outer diameter of the electric plug 74. The reduced diameter portion 75 is fixed by being inserted through the through hole 65 of the first handle body 60. Specifically, a flange 76 is provided between the reduced diameter portion 75 and the connecting portion 71, and a part of the connector support portion 64 is sandwiched between the flange 76 and the electric plug 74, so that the electric connector 7 is mechanically fixed to the first handle body 60 (connector support portion 64). The fixing method of the electric connector 7 and the first handle body 60 is not limited to this, and they may be fixed by means of adhesive or the like.

The connecting portion 71 and the electric plug 74 arranged on both sides of the reduced diameter portion 75 in the vertical direction B cannot be inserted through the through hole 65 of the first handle body 60. The connecting portion 71 and the electric plug 74 engage with the openings at both ends of the through hole 65 in the vertical direction B, so that the movement range of the electric connector 7 in the vertical direction B is restricted to a predetermined range.

The through hole 65 is a through hole through which the reduced diameter portion 75 of the electric connector 7 is inserted. The length of the through hole 65 in the vertical direction B may be slightly shorter than the length of the reduced diameter portion 75 in the vertical direction B.

The plug socket 66 is cylindrical and surrounds the energized plug 74 of the electrical connector 7. A gap is provided between the energized plug 74 of the electrical connector 7 and the plug socket 66 to allow for attachment of a power cable (active cord).

The electrical connector (connecting member, plug) 7 can be connected to a high-frequency power supply device (not shown) via a power cable, and is electrically and physically connected to the proximal end connection portion 2b. The proximal end connection portion 2b connected to the electrical connector 7 does not have a coil sheath 13, but has a wire 21 and a pipe 22 arranged therein. The electrical connector 7 can supply high-frequency current supplied from the high-frequency power supply device to the forceps 4 via the pipe 22 and wire 21.

The coil spring support portion 67 has a circular recess on its inner circumferential surface, and the recess supports the coil spring 9.

The coil sheath support portion 69 supports the proximal end of the coil sheath 13 so that the proximal end of the coil sheath 13 can rotate around the longitudinal axis and does not advance or retract relative to the handle body 6 along the longitudinal axis direction A. As shown in FIG. 3, the distal end of the pipe 22 overlaps with the proximal end of the coil sheath 13 inside the handle body 6. The electrical connector 7 is disposed on the distal side A1 of the pipe 22. In the longitudinal axis direction A of the wire 21, the proximal end 13p of the coil sheath 13 is disposed between the distal end 22b of the pipe 22 and the electrical connector 7.

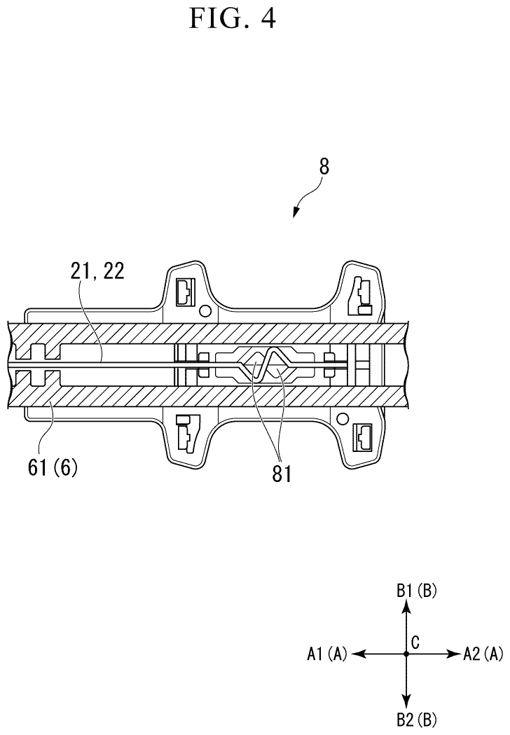

FIG. 4 is a cross-sectional view of the slider 8.

The slider 8 is attached so as to be able to advance and retract in the longitudinal axis direction A along the second handle body 61. The slider 8 can advance and retract along the longitudinal axis relative to the second handle body 61. The proximal end of the wire 21 and the proximal end of the pipe 22 are connected to the slider 8. The wire 21 and the pipe 22 advance and retract when the surgeon advances and retracts the slider 8 relative to the second handle body 61. The wire 21 and the pipe 22 rotate when the surgeon rotates the second handle body 61 and the slider 8 around the longitudinal axis relative to the first handle body 60.

The slider 8 has an operating wire support portion 81 that supports the wire 21 and the pipe 22. The wire 21 and the pipe 22 are fixed to the operating wire support portion 81. Specifically, the wire 21 and the pipe 22 are fixed to the operating wire support portion 81 by passing between the two separated operating wire support portions 81 in an S-shaped serpentine shape. It is not necessary to fix both the wire 21 and the pipe 22 to the slider 8. For example, the proximal end of the wire 21 may be fixed to any position on the pipe 22, and only the pipe 22 may be fixed to the slider 8. In addition, the pipe 22 does not necessarily have to be a pipe in the strict sense, and may be any member that functions as the transmission member 2A, and may be a pipe with a spiral groove or a highly rigid metallic coil tube.

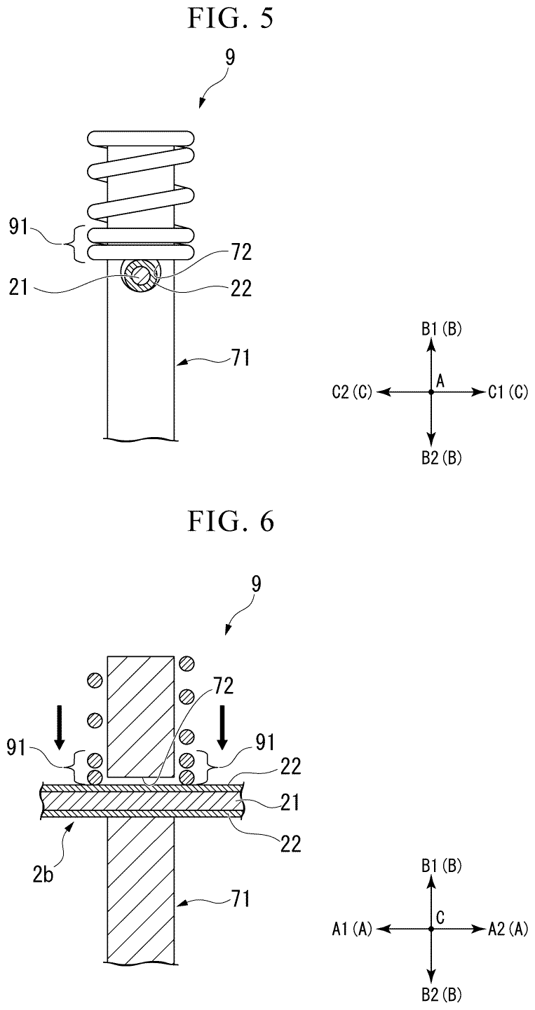

FIG. 5 is a diagram showing a coil spring 9.

The coil spring (elastic member, elastic body) 9 electrically connects the pipe 22 (the wire 21 passing through the pipe 22) and the electrical connector 7 by applying a biasing force to the pipe 22. The coil spring 9 is supported by the coil spring support portion 67 so as to be elastically deformable along the vertical direction B. That is, the coil spring 9 is supported by the coil spring support portion 67 (recess) so as to be elastically deformable along the extension direction of the electrical connector 7. The coil spring 9 is a conductive compression spring, and an elastic force acts in the direction of extension along the vertical direction B. Specifically, the coil spring 9 comes into direct contact with the pipe 22 to press the pipe 22 against the electrical connector 7 (the connection portion 71 of the electrical connector 7). The coil spring 9 biases the pipe 22 toward the electrical connector 7 (the connection portion 71 of the electrical connector 7) and presses the pipe 22 against the inner surface of the insertion passage 72 of the electrical connector 7. The coil spring 9 is in a compressed state and in contact with the pipe 22, and with the pipe 22 pressed against the inner surface of the insertion passage 72 of the electrical connector 7, the pipe 22 and the wire 21 passing through the pipe 22 can advance and retract through the insertion passage 72 of the electrical connector 7 relative to the electrical connector 7, and can also rotate. In other words, with the pipe 22 pressed against the electrical connector 7, the pipe 22 and the wire 21 passing through the pipe 22 can advance and retract (slide) relative to the electrical connector 7 along the longitudinal axis of the pipe 22, and can rotate (slide) relative to the electrical connector 7 in the direction around the longitudinal axis of the pipe 22.

In this embodiment, the upper end (second end) 70 of the electrical connector 7 is inserted into the coil spring 9. The coil spring 9 is in a compressed state and is disposed between the bottom surface of the recess of the coil spring support portion 67 and the pipe 22. The coil spring 9 presses the pipe 22, which passes through the passage 72, against the lower side B2 by means of a biasing force, thereby electrically connecting the pipe 22 (the wire 21 passing through the pipe 22) to at least a part of the passage 72 of the electrical connector 7.

FIG. 6 is a cross-sectional view of the coil spring 9.

In this embodiment, a seat turn 91 is provided at the end of the lower side B2 of the coil spring 9 (the part that contacts the pipe 22). The seat turn 91 is, for example, 1 to 2 turns. Because the coil pitch of the seat turn 91 is narrow, the pipe 22 does not enter the seat turn 91. Specifically, the coil pitch of the seat turn 91 is smaller than the outer diameter of the pipe 22, which passes through the passage 72. Therefore, even if the pipe 22 rotates, the pipe 22 can be prevented from entering the inside of the coil spring 9 from the end of the lower side B2 of the coil spring 9, and the smooth rotational movement of the pipe 22 is not hindered. The upper end of the coil spring 9 may also be provided with a narrow-pitch seat winding.

In this embodiment, the upper end 70 of the electrical connector 7 is inserted into the coil spring 9. Therefore, the coil spring 9 can press the pipe 22 against the lower side B2 from both sides, sandwiching the electrical connector 7. Therefore, a stable electrical connection can be achieved between the pipe 22 (the wire 21 passing through the pipe 22) and the electrical connector 7. In addition, the ground contact area between the pipe 22 and the electrical connector 7 is increased. Therefore, an increase in the electrical resistance value caused by a decrease in the ground contact area between the pipe 22 and the electrical connector 7 can be prevented. At this time, if the elastic force of the coil spring 9 is too strong, the pipe 22 and the insertion passage 72 may come into contact too strongly, which may affect the advancing and retracting movement of the pipe 22 and the advancing and retracting movement of the slider 8. Therefore, it may be desirable that the coil spring 9 has an elastic force that does not provide excessive resistance to the advancement and retraction of the slider 8. Similarly, it may be desirable that the coil spring 9 has an elastic force that does not provide excessive resistance to the rotational movement of the pipe 22 and the rotational operation of the slider 8.

In order to position the forceps 4 at an appropriate treatment position, the surgeon advances and retracts or rotates the forceps 4 by grasping the slider 8 and advancing and retracting it or rotating it. Even when the slider 8 advances and retracts relative to the second handle body 61 or the second handle body 61 and the slider 8 rotate relative to the first handle body 60, the coil spring 9 presses the pipe 22 against the electrical connector 7, thereby reliably ensuring electrical connection between the pipe 22 and the wire 21 passing through the pipe 22 and the electrical connector 7.

Even if the slider 8 is rotated around the longitudinal axis of the sheath 1, the electrical connector 7 does not rotate around the longitudinal axis of the sheath 1, so the power cable (active cord) connected to the electrical connector 7 does not get tangled in the operating portion 5.

The surgeon applies high-frequency current to the proximal end connection portion 2b (wire 21 and pipe 22). Regardless of which direction perpendicular to the longitudinal axis direction A of the operating portion 5 faces vertically downward, at least a part of the insertion passage 72 comes into contact with the pipe 22 so that it can be electrically connected. Therefore, high-frequency current is applied from the electrical connector 7 to the wire 21 and pipe 22 regardless of the position of the operating portion 5, and the forceps 4 can be used to stop bleeding or to make an incision.

According to the endoscope treatment tool 100 of this embodiment, regardless of the position of the operating portion 5, electrical connection between the electrical connector 7 to which high-frequency current is supplied and the wire 21 and pipe 22 connected to the forceps 4 are reliably secured.

The first embodiment of the present disclosure has been described above in detail with reference to the drawings, but the specific configuration is not limited to this embodiment, and design modifications within the scope of the present disclosure are also included. In addition, the components shown in the above-mentioned embodiment and modifications can be configured by appropriately combining them.

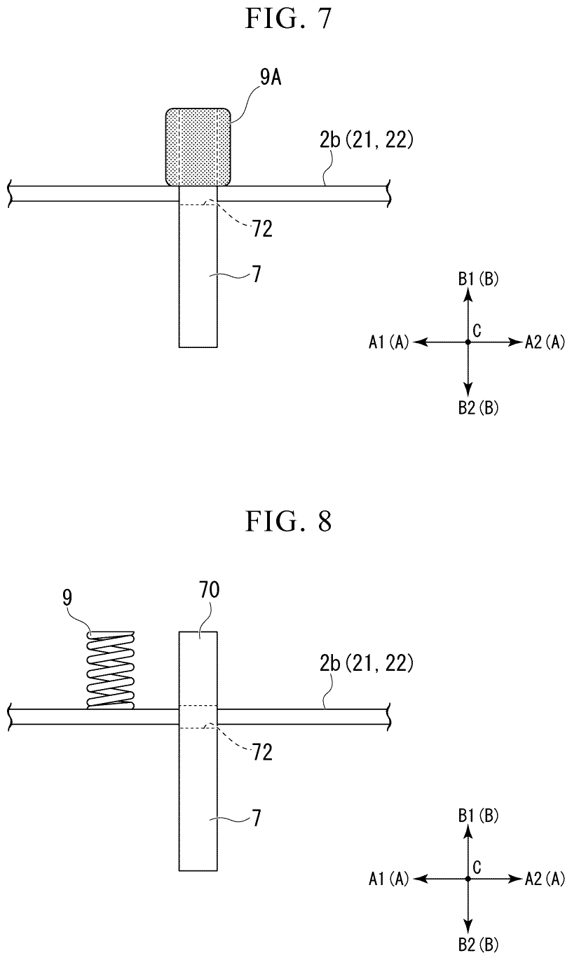

FIG. 7 is a diagram showing an elastic spring 9A, which is a modification of the coil spring 9.

The elastic spring (elastic member, elastic body) 9A is a member having elasticity such as rubber. Like the coil spring 9, the elastic spring 9A electrically connects the pipe 22 (the wire 21 passing through the pipe 22) and the electrical connector 7 by applying a biasing force to the pipe 22. In other words, the elastic member that electrically connects the pipe 22 and the electrical connector 7 may be a conductor such as the coil spring 9, or a non-conductor such as the elastic spring 9A.

FIG. 8 is a diagram showing another aspect of the coil spring 9.

The coil spring 9 does not have to be arranged on the outer periphery of the upper end 70 of the electrical connector 7. As shown in FIG. 8, the coil spring 9 may be disposed at a position spaced apart from the electrical connector 7, and may electrically connect the pipe 22 (the wire 21 passing through the pipe 22) and the electrical connector 7 by exerting a biasing force on the pipe 22.

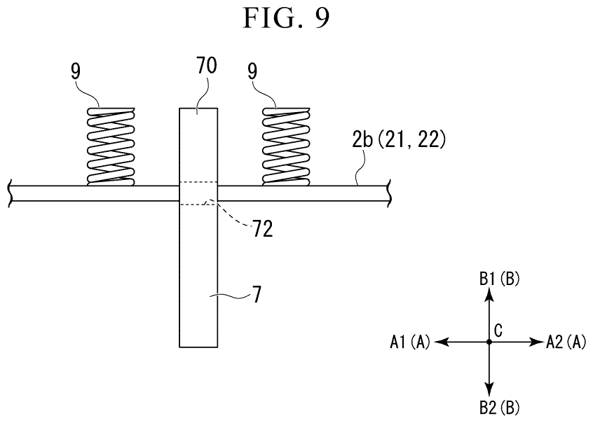

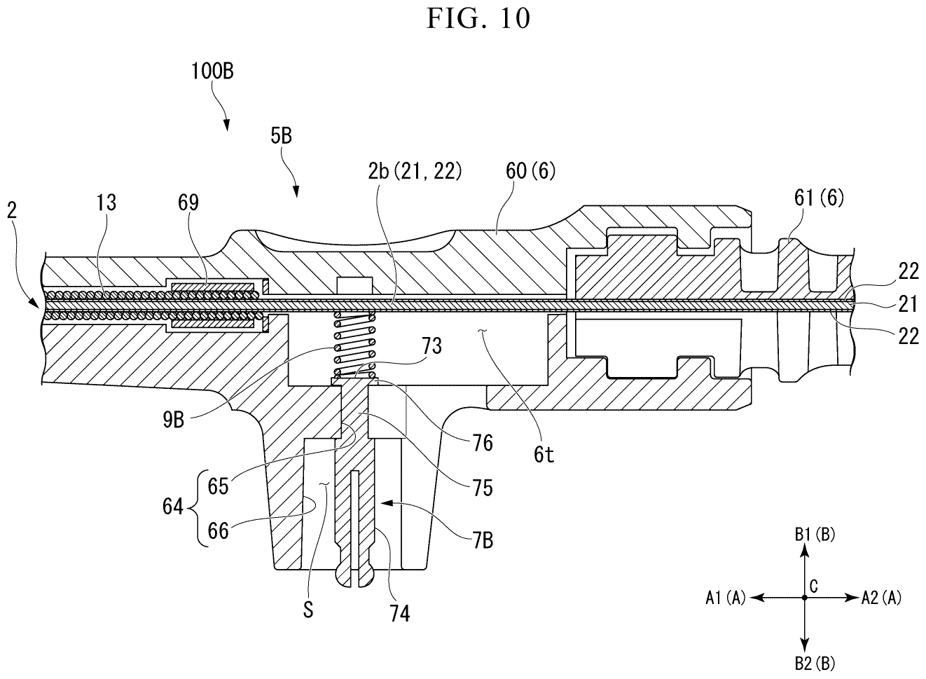

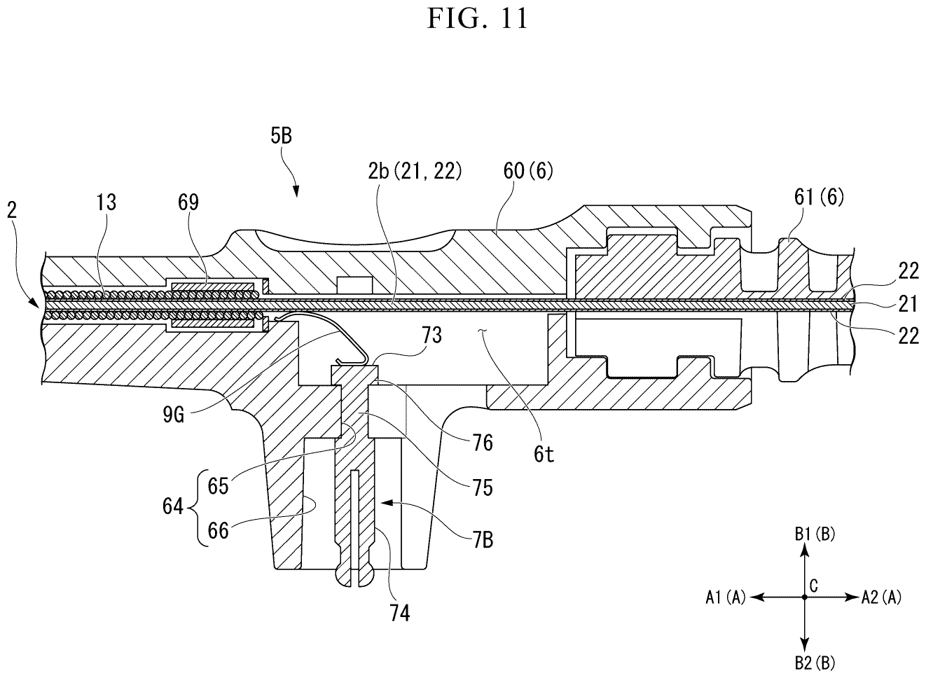

FIG. 9 is a diagram showing another aspect of the coil spring 9.

Multiple coil springs 9 may be disposed. The multiple coil springs 9 can suitably connect the pipe 22 (the wire 21 passing through the pipe 22) and the electrical connector 7.

### Second Embodiment

An endoscope treatment tool 100B according to a second embodiment of the present disclosure will be described with reference to FIG. 10. In the following description, components common to those already described will be given the same reference numerals and will not be described again.

### [Endoscope Treatment Tool 100B]

The endoscope treatment tool 100B (also called treatment tool 100B) is a hemostatic forceps that cauterizes the affected area to stop bleeding. The treatment tool 100B includes a sheath 1, a long member 2, a transmission member 2A, a support member 3, forceps (jaw) 4, and an operating portion 5B.

FIG. 10 is a cross-sectional view of the operating portion 5B. The operating portion (handle, handle assembly) 5B is provided on the proximal end side A2 of the sheath 1. The operating portion 5B comprises a handle body 6, an electrical connector 7B, a slider 8, and a coil spring 9B. In this embodiment, the electrical connector 7B is attached to the handle body 6.

The electrical connector (connecting member, plug) 7B can be connected to a high-frequency power supply device (not shown) via a power cable, and is electrically connected to the pipe 22 (the proximal end of the wire 21) via a coil spring 9B. The proximal end connection portion 2b connected to the electrical connector 7B does not have a coil sheath 13, and the wire 21 and the pipe 22 are arranged therein. The electrical connector 7B can supply high-frequency current supplied from the high-frequency power supply device to the forceps 4 via the pipe 22 and the wire 21. The electrical connector 7B is arranged at a position separated from the wire 21 and the pipe 22.

The handle body 6 has a connector support portion 64. The connector support portion 64 is provided on the first handle body 60. The electrical connector 7B is supported by the connector support portion 64, and is formed in a substantially cylindrical shape extending in a direction intersecting the longitudinal axis of the pipe 22. In addition, “approximately cylindrical” includes not only a strict cylindrical shape but also a shape close to a cylindrical shape. In this embodiment, the electrical connector 7B extends in the vertical direction B.

The electrical connector 7B extends in a direction intersecting the longitudinal axis of the pipe 22. The electrical connector 7B has an engagement portion 73, an electrical plug 74, and a reduced diameter portion 75. The engagement portion 73 is provided at the upper end of the electrical connector 7B and engages with the coil spring 9B. The electrical connector 7B extends between the electrical plug (first end) 74 and the engagement portion (second end) 73.

The connector support portion 64 supports the electrical connector 7B and has a plug socket 66 that covers the electrical plug 74 with a gap S around it. The connector support portion 64E has a through hole 65 that connects the gap S to the internal space 6t. With the electrical connector 7E inserted into the through hole 65, the electrical connector 7B is positioned relative to the connector support portion 64.

The electrical connector 7B is supported by the connector support portion 64. Specifically, the electrical connector 7B is fixed to the connector support portion 64 by providing a flange 76 on the engagement portion 73 and sandwiching a part of the connector support portion 64 (the edge of the through hole 65) between the flange 76 and the conductive plug 74.

The flange 76 is a protrusion that protrudes radially outward from the electrical connector 7E. The flange 76 is disposed between the conductive plug 74 and the pipe 22.

The coil spring (elastic member, elastic body) 9B electrically connects the pipe 22 (the wire 21 passing through the pipe 22) and the electrical connector 7B. The coil spring 9B is sandwiched between the pipe 22 and the electric connector 7B (more specifically, the engaging portion 73 or the flange 76) in the internal space 6t of the handle body 6 in a compressed state. The coil spring 9B is a conductive compression spring, and an elastic force acts in the direction in which the electric connector 7B extends. Specifically, the coil spring 9B directly contacts the pipe 22 and presses the pipe 22 in the direction opposite to the direction facing the engaging portion (second end) 73 of the electric connector 7B. That is, the coil spring 9B presses the pipe 22 from the direction intersecting the longitudinal axis of the pipe 22. When the pipe 22 is pressed by the coil spring 9B, the pipe 22 is elastically deformed, and a restoring force that restores the pipe 22 to its original shape is generated in the pipe 22, and the pipe 22 presses back the coil spring 9B. This allows the pipe 22 and the coil spring 9B to always maintain a state of contact with each other. In addition, the proximal end connection portion 2b may be always in contact with the wire 21 and the inner peripheral surface of the pipe 22. When the coil spring 9B presses the pipe 22 in the opposite direction to the direction facing the engaging portion (second end) 73 of the electrical connector 7B, the pipe 22 is slightly bent in the opposite direction. In this state, the pipe 22 and the wire 21 passing through the pipe 22 can advance and retract with respect to the electrical connector 7B, and can rotate. In other words, when the pipe 22 is pressed against the electrical connector 7B in a direction the pipe 22 is away from the electrical connector 7B, the pipe 22 and the wire 21 passing through the pipe 22 can advance and retract (slide) with respect to the electrical connector 7B in the direction along the longitudinal axis of the pipe 22, and can rotate (slide) with respect to the electrical connector 7B in the direction around the longitudinal axis of the pipe 22.

In this embodiment, the coil spring (elastic member, elastic body) 9B electrically connects the pipe 22 and the wire 21 passing through the pipe 22 to the electrical connector 7B by applying a biasing force to the pipe 22. The coil spring 9B is supported in the internal space 6t of the handle body 6 so as to be elastically deformable in the vertical direction B. The coil spring 9B is a conductive compression spring, and an elastic force acts in the direction of extension along the vertical direction B.

In this embodiment, the coil spring 9B is arranged in a compressed state in the space between the pipe 22 and the engaging portion 73 of the electrical connector 7B. The coil spring 9B presses the pipe 22 to the upper side B1 by the biasing force, thereby electrically connecting the pipe 22 and the wire 21 passing through the pipe 22 to the engaging portion 73 of the electrical connector 7B. Note that a narrow-pitch seat coil may be provided at the upper and lower ends of the coil spring 9B.

According to the endoscope treatment tool 100B of this embodiment, the electrical connection between the electrical connector 7 to which high-frequency current is supplied and the wire 21 and pipe 22 connected to the forceps 4 are reliably secured regardless of the position of the operating portion 5B.

The second embodiment of the present disclosure has been described above in detail with reference to the drawings, but the specific configuration is not limited to this embodiment, and design modifications within the scope of the present disclosure are also included. In addition, the components shown in the above-mentioned embodiment and modifications can be configured in any suitable combination.

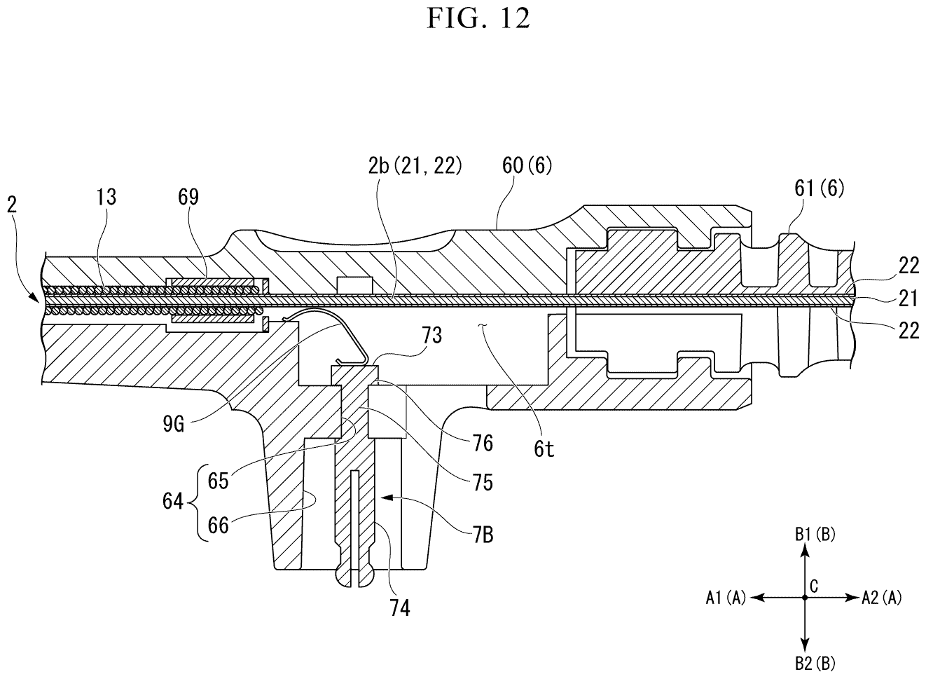

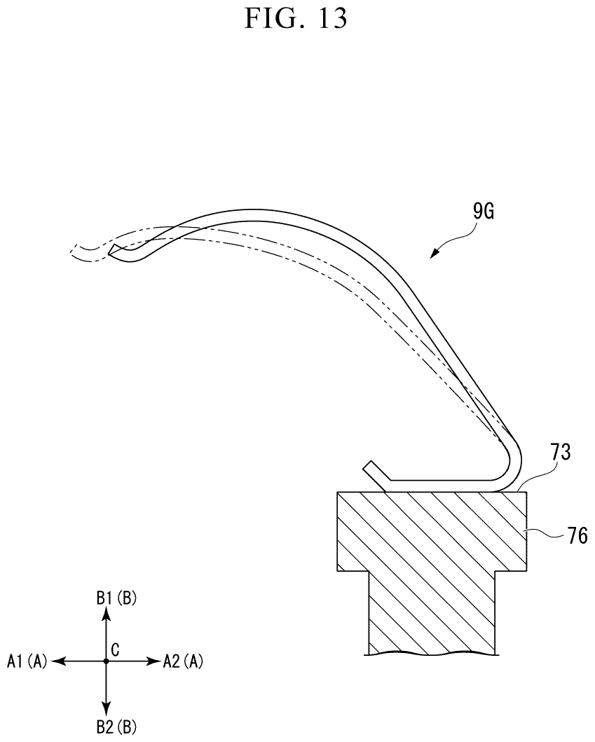

FIGS. 11 to 13 are diagrams showing a leaf spring 9G, which is a modification of the coil spring 9B.

The leaf spring 9G electrically connects the pipe 22 (the wire 21 passing through the pipe 22) to the electrical connector 7B by applying a biasing force to the pipe 22. The leaf spring 9G is placed in a space between the pipe 22 and the engaging portion 73 of the electrical connector 7 in a compressed state. The leaf spring 9G electrically connects the pipe 22 (the wire 21 passing through the pipe 22) to the engaging portion 73 of the electrical connector 7 by pressing the pipe 22 against the upper side B1 with a biasing force.

### Third Embodiment

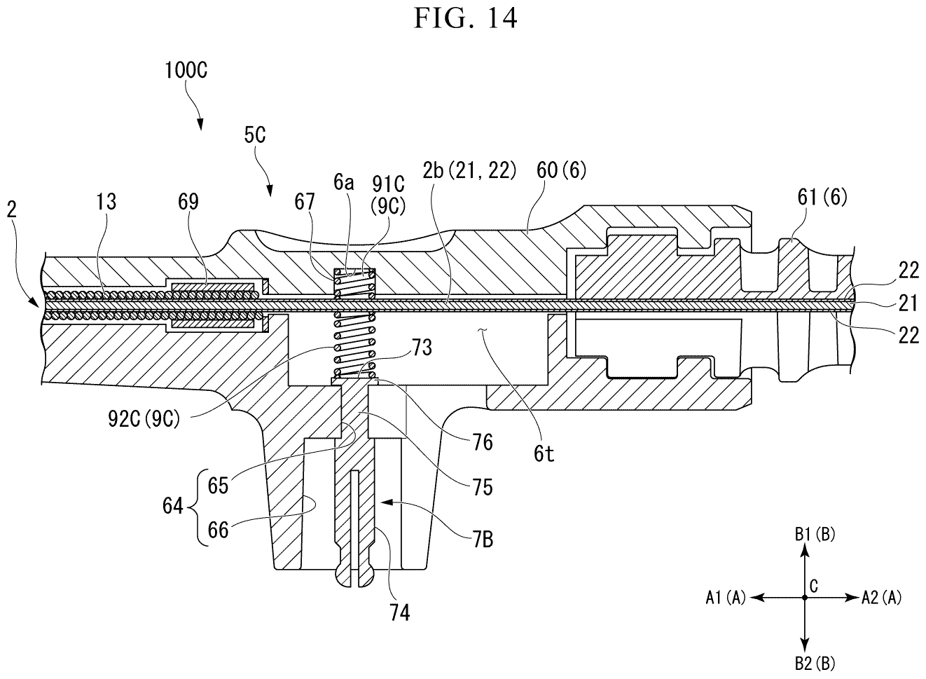

The endoscope treatment tool 100C according to the third embodiment of the present disclosure will be described with reference to FIG. 14. In the following description, the same components as those already described will be denoted by the same reference numerals, and a duplicated description will be omitted.

[Endoscope Treatment Tool 100C]The endoscope treatment tool 100C (also called treatment tool 100C) is a hemostatic forceps that cauterize the affected area to stop bleeding. The treatment tool 100C comprises a sheath 1, a long member 2, a transmission member 2A, a support member 3, forceps (jaw) 4, and an operating portion 5C.

FIG. 14 is a cross-sectional view of the operating portion 5C.

The operating portion (handle, handle assembly) 5C is provided on the proximal end side A2 of the sheath 1. The operating portion 5C comprises a handle body 6, an electrical connector 7B, a slider 8, and a coil spring 9C. In this embodiment, the electrical connector 7B is attached to the handle body 6.

The coil spring (elastic member, elastic body) 9C electrically connects the pipe 22 to the electrical connector 7B by applying a biasing force to the pipe 22. The coil spring 9C has a first coil spring 91C and a second coil spring 92C. The first coil spring 91C is supported by the coil spring support portion 67 in the internal space 6t of the handle body 6 so as to be elastically deformable along the vertical direction B. That is, the first coil spring 91C is a conductive compression spring, and is supported by the coil spring support portion 67 (recess). The first coil spring 91C is disposed in a compressed state between the coil spring support portion 67 and the pipe 22. Specifically, the first coil spring 91C comes into direct contact with the pipe 22 and presses the pipe 22 against the engaging portion (second end) 73 of the electrical connector 7B. That is, the first coil spring 91C presses the pipe 22 from a direction intersecting the longitudinal axis of the pipe 22. The second coil spring 92C is a conductive compression spring, and is disposed in a compressed state between the electric connector 7B (more specifically, the engaging portion 73 or the flange 76) and the pipe 22. Specifically, the second coil spring 92C directly contacts the pipe 22 and presses the pipe 22 in a direction opposite to the direction in which the engaging portion (second end) 73 of the electric connector 7B faces. That is, the second coil spring 92C presses the pipe 22 from a direction intersecting the longitudinal axis of the pipe 22. Since the first coil spring 91C presses the pipe 22 in a direction opposite to the direction in which the second coil spring 92C presses the pipe 22, the second coil spring 92C and the pipe 22 can be preferably maintained in a state in which they may be always in contact with each other. In the proximal end connection portion 2b, the wire 21 and the inner peripheral surface of the pipe 22 may be always in contact with each other. When both the first coil spring 91C and the second coil spring 92C are pressing against the pipe 22, the pipe 22 and the wire 21 passing through the pipe 22 can advance and retract relative to the electrical connector 7B, and can rotate. That is, when the pipe 22 is pressed against the second coil spring 92C, the pipe 22 and the wire 21 passing through the pipe 22 can advance and retract (slide) relative to the electrical connector 7B in the direction along the longitudinal axis of the pipe 22, and can rotate (slide) relative to the electrical connector 7B in the direction around the longitudinal axis of the pipe 22.

In this embodiment, the coil spring 9C is disposed in the space between the upper inner wall 6a of the handle body 6 and the engaging portion 73 of the electrical connector 7 in the internal space 6t of the handle body 6. The pipe 22 penetrates the coil spring 9C in the longitudinal axis direction A. That is, the coil spring 9C is disposed on both sides of the pipe 22 in the up-down direction B. The coil spring 9C may be a single spring, or may be divided into two, each arranged on either side of the pipe 22 in the vertical direction B.

Even if the pipe 22 moves in the vertical direction B, the coil spring 9C can maintain contact with the pipe 22 due to the biasing force, electrically connecting the pipe 22 to the engagement portion 73 of the electrical connector 7. In addition, a narrow-pitch seat coil may be provided at the upper and lower ends of the first coil spring 91C and the second coil spring 92C.

According to the endoscope treatment tool 100C of this embodiment, the electrical connection between the electrical connector 7 to which high-frequency current is supplied and the wire 21 and pipe 22 connected to the forceps 4 are reliably secured, regardless of the position of the operating portion 5C.

The third embodiment of the present disclosure has been described above in detail with reference to the drawings, but the specific configuration is not limited to this embodiment, and design changes within the scope of the present disclosure are also included. In addition, the components shown in the above-mentioned embodiments and modifications can be appropriately combined to form a configuration.

### Fourth Embodiment

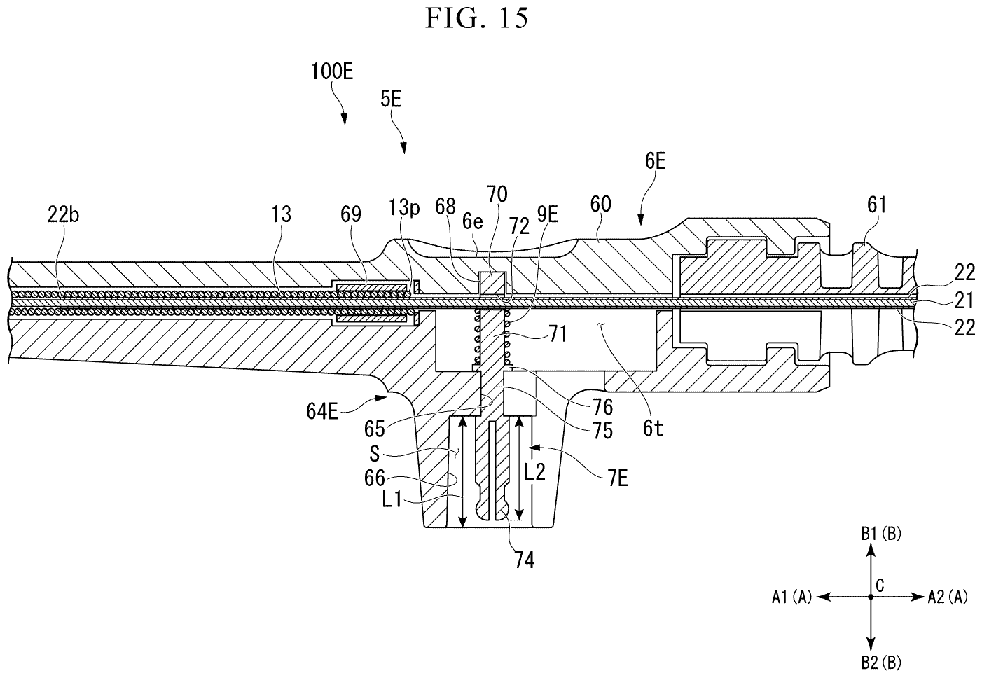

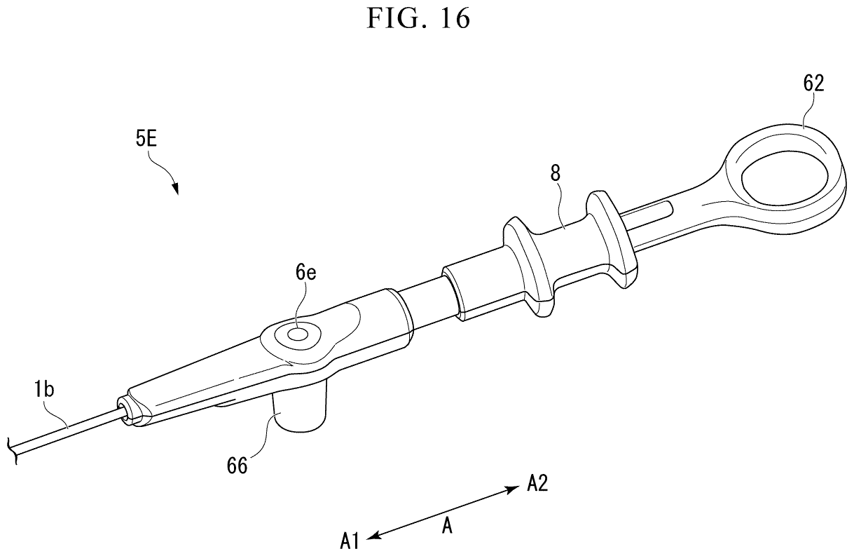

An endoscope treatment tool 100E according to a fourth embodiment of the present disclosure will be described with reference to FIGS. 15 and 16. In the following description, components common to those already described will be given the same reference numerals and a redundant description will be omitted.

### [Endoscope Treatment Tool 100E]

The endoscope treatment tool 100E (also called the treatment tool 100E) is a hemostatic forceps that cauterizes the affected area to stop bleeding. The treatment tool 100E includes a sheath 1, a long member 2, a transmission member 2A, a support member 3, a forceps (jaw) 4, and an operating portion 5E.

FIG. 15 is a cross-sectional view of the operating portion 5E. FIG. 16 is a perspective view of the operating portion 5E.

The operating portion (handle, handle assembly) 5E is provided on the proximal end side A2 of the sheath 1. The operating portion 5E includes a handle body 6E, an electrical connector 7E, a slider 8, and a coil spring 9E. In this embodiment, the electrical connector 7E is attached to the handle body 6E.

The handle body 6E has an internal space 6s through which the wire 21 can be inserted. The wire 21 passes through the internal space 6s of the sheath 1 and the handle body 6, and extends to the slider 8. The handle body 6E has a thumb ring 62, a connector support portion 64E, and a coil sheath support portion 69.

The handle body 6E has a first handle body 60 and a second handle body 61.

The second handle body 61 is connected to the proximal end of the first handle body 60. The first handle body 60 and the second handle body 61 are connected to be capable of relative rotation about a rotation axis extending in the longitudinal axis direction A. The internal space 6s is a continuous space formed in the first handle body 60 and the second handle body 61. The internal space 6s has an internal space 6t formed in the first handle body 60 and an internal space 6u formed in the second handle body 61.

As shown in FIG. 15, the distal end of the pipe 22 overlaps with the proximal end of the coil sheath 13 in the handle body 6E, and the electrical connector 7E is disposed on the distal side Al of the pipe 22. In the longitudinal axis direction A of the wire 21, the proximal end 13p of the coil sheath 13 is disposed between the distal end 22b of the pipe 22 and the electrical connector 7E.

The electrical connector 7E is a conductive member extending in a direction intersecting the longitudinal axis of the pipe 22, and is formed in a generally cylindrical shape. Note that “approximately cylindrical” includes shapes close to a cylindrical shape as well as a strict cylindrical shape. In this embodiment, the electrical connector 7E extends in the vertical direction B.

The electrical connector 7E has an upper end 70, a connecting portion 71, an electrical plug 74, a reduced diameter portion 75, and a flange 76. As shown in FIG. 15, the electrical connector 7E extends between the electrical plug (first end) 74 and the upper end (second end) 70. The connecting portion 71 and the reduced diameter portion 75 are located between the electrical plug (first end) 74 and the upper end (second end) 70. The connecting portion 71 has an insertion passage 72 extending in a direction intersecting the direction in which the electrical connector 7E extends.

The connector support portion 64E is provided on the first handle body 60. The connector support portion 64E supports the electric connector 7E and has a plug socket 66 that covers the electric plug 74 with a gap S around it. The connector support portion 64E has a through hole 65 that connects the gap S to the internal space 6t. When the electric connector 7E is inserted into the through hole 65, the electric connector 7E is positioned relative to the connector support portion 64E.

The handle body 6E has a fitting portion 68. The fitting portion 68 is a circular recess on its inner circumferential surface, and supports the upper end portion 70 of the electric connector 7E. The gap between the upper end portion 70 of the electric connector 7E and the fitting portion 68 is narrow, preventing the electric connector 7E from wobbling.

The electrical connector 7E (specifically, the conductive plug 74) can be connected to a high-frequency power supply device (not shown) via a power cable, and is electrically and physically connected to the proximal end connection portion 2b. The proximal end connection portion 2b connected to the electrical connector 7E does not have a coil sheath 13, but has a wire 21 and a pipe 22 arranged therein. The electrical connector 7E can supply high-frequency current supplied from the high-frequency power supply device to the forceps 4 via the pipe 22 and the wire 21.

The electrical connector 7E is supported by the connector support portion 64 and the fitting portion 68. Specifically, the electrical connector 7E is fixed to the connector support portion 64E by clamping a part of the connector support portion 64 (the edge of the through hole 65) between the flange 76 and the conductive plug 74. The electrical connector 7E extends in a direction intersecting the longitudinal axis of the wire 21. The flange 76 is arranged between the conductive plug 74 and the pipe 22.

The flange 76 is provided between the conductive plug 74 and the upper end 70. Specifically, it is provided between the reduced diameter portion 75 and the connecting portion 71 of the electrical connector 7E. The flange 76 is a protrusion that protrudes radially outward from the electrical connector 7E.

The coil spring (elastic member, elastic body) 9E electrically connects the pipe 22 (the wire 21 passing through the pipe 22) and the electrical connector 7E by applying a biasing force to the pipe 22. A part of the electrical connector 7E (the connecting portion 71) is inserted through the coil spring 9E. This prevents the coil spring 9E from shifting in the longitudinal axis direction of the pipe 22, and suppresses the occurrence of electrical failure. The coil spring 9E is sandwiched between the pipe 22 and the electrical connector 7E (more specifically, the flange 76) in a compressed state in the internal space 6t of the handle body 6E. The coil spring 9E is a conductive compression spring, and an elastic force acts in the direction in which the electrical connector 7E extends. Specifically, the coil spring 9E comes into direct contact with the pipe 22 and presses the pipe 22 against the electrical connector 7E (the inner surface of the insertion passage 72 of the electrical connector 7E). That is, the coil spring 9E presses the pipe 22 from a direction intersecting the longitudinal axis of the pipe 22. When the pipe 22 is pressed by the coil spring 9E, the pipe 22 is elastically deformed, and a restoring force that restores the pipe 22 to its original shape is generated in the pipe 22, and the pipe 22 presses back against the coil spring 9E. This allows the pipe 22 and the coil spring 9E to maintain a constant contact state. The proximal end connection portion 2b may be always in contact with the wire 21 and the inner peripheral surface of the pipe 22. The coil spring 9E urges the pipe 22 toward the upper end 70 of the electrical connector 7E. When the coil spring 9E presses the pipe 22 against the upper end 70 of the electrical connector 7E, the pipe 22 is slightly bent toward the upper end 70 of the electrical connector 7E. When the coil spring 9E is in a compressed state and in contact with the pipe 22 and the pipe 22 is pressed against the inner surface of the insertion passage 72 of the electrical connector 7E, the pipe 22 and the wire 21 passing through the pipe 22 can advance and retract through the insertion passage 72 of the electrical connector 7E relative to the electrical connector 7E, and can also rotate. In other words, when the pipe 22 is pressed against the electrical connector 7E, the pipe 22 and the wire 21 passing through the pipe 22 can advance and retract (slide) relative to the electrical connector 7E in a direction along the longitudinal axis of the pipe 22, and can rotate (slide) relative to the electrical connector 7E in a direction around the longitudinal axis of the pipe 22.

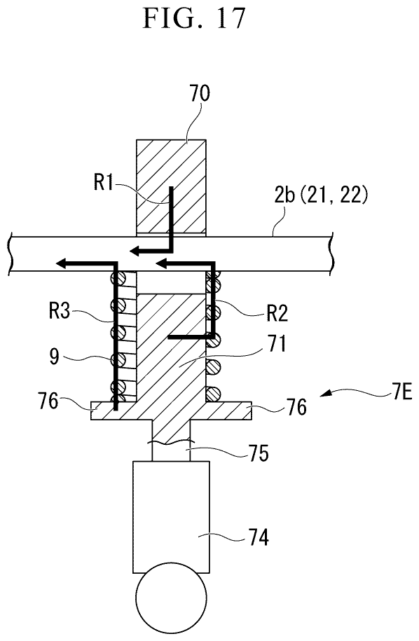

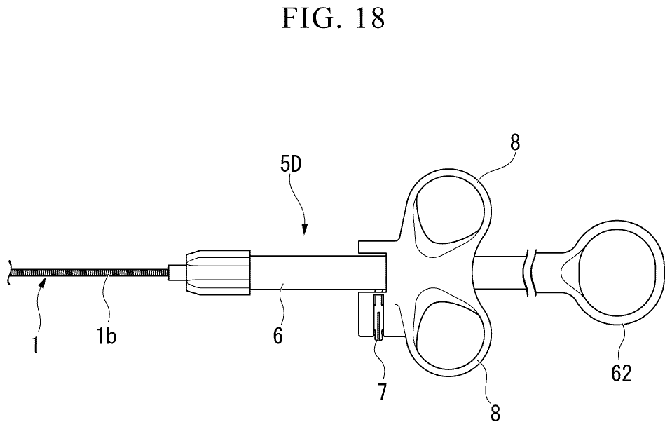

FIG. 17 is a diagram showing the current path from the electrical connector 7E to the pipe 22.

There are three current paths from the electrical connector 7E to the pipe 22 (proximal end connection portion 2b): path R1, path R2, and path R3. Path R1 is from the electrical connector 7E (upper end 70) to the pipe 22 (proximal end connection portion 2b). Path R2 is from the coupling portion 71 of the electrical connector 7E to the pipe 22 (proximal end connection portion 2b) via the coil spring 9E. Path R3 is from the flange 76 of the electrical connector 7E to the pipe 22 (proximal end connection portion 2b) via the coil spring 9E. In particular, in path R3, the coil spring 9E and the flange 76 may be always in contact. By configuring the electrical connector 7E in this way, it is possible to reduce the occurrence of electrical failure. This makes it possible to realize stable electrical conduction while reducing the sliding resistance of the wire 21 passing through the pipe 22 and the electrical connector 7E, such as the rotation and advancement and retract, while also reducing the sliding resistance. The upper and lower ends of the coil spring 9E may be provided with narrow-pitch seat turns.

The length L1 of the plug socket 66 in the vertical direction B is longer than the length L2 of the plug 74 in the vertical direction B. Therefore, the plug 74 is unlikely to be connected to the power cable when tilted in the vertical direction B. Furthermore, the upper fitting portion 68 prevents the electric connector 7E from wobbling, so the power cable and the plug 74 are likely to be connected straight along the vertical direction B. As a result, it is possible to prevent the electric connector 7E from bending, which would make it difficult to connect to the power cable.

As shown in FIGS. 15 and 16, the handle body 6E has a concave surface (indicator) 6e that can be held with the thumb or the like. In the longitudinal axis direction A of the operating portion 5E, the electric connector 7E and the plug socket 66 are fixed at a position that overlaps with the concave surface 6e. Therefore, the surgeon can easily hold the handle body 6E.

The endoscope treatment tool 100E according to this embodiment can reliably ensure electrical connection between the electrical connector 7 to which high-frequency current is supplied and the wire 21 and pipe 22 connected to the forceps 4, regardless of the position of the operating portion 5E, while allowing the wire 21 to rotate and advance and retract relative to the electrical connector 7E.

The fourth embodiment of the present disclosure has been described above in detail with reference to the drawings, but the specific configuration is not limited to this embodiment and includes design changes and the like that do not deviate from the gist of the present disclosure. In addition, the components shown in the above-mentioned embodiments and modifications can be appropriately combined.

FIG. 18 shows an operating portion 5D, which is a modification of the operating portion 5, operating portion 5B, and operating portion 5C. In the operating portion 5D, the electrical connector 7 is attached to the slider 8. Even when the electrical connector 7 is attached to the slider 8, the electrical connection between the electrical connector 7 and the elongated member 2 is reliably ensured by the coil spring (elastic member, elastic body).

FIG. 19 is a diagram showing the treatment portion (end effector) 110. The following provides additional information regarding the treatment portion (end effector) 110.

The support member 3 has a cylindrical support body 30, a frame 31, a connecting member 32, a rod 33, and a link mechanism 34. The support body 30 is attached to the distal end 1a of the sheath 1 and is rotatable around a rotation axis along the longitudinal axis direction A. The frame 31 is attached to the support body 30 and protrudes from the distal end 1a of the sheath 1 to the distal side A1. The connecting member 32 connects the distal end of the wire 21 to the rod 33. The rod 33 is a rod-shaped member and protrudes from the distal end 1a of the sheath 1 to the distal side A1. The rod 33 advances and retracts in the longitudinal axis direction A along the frame 31. The distal end of the rod 33 is connected to the link mechanism 34. The proximal end of the rod 33 is connected to the connecting member 32. The link mechanism 34 converts the advancement and retract movement of the rod 33 into the opening and closing movement of the forceps 4. The link mechanism 34 has a first link 341 and a second link 342.

The forceps 4 have a pin 40, a first forceps piece 41, and a second forceps piece 42. The first forceps piece 41 and the second forceps piece 42 have a gripping portion 4a that grips tissue. The pin 40 is fixed to the frame 31. The first forceps piece 41 and the second forceps piece 42 are connected to be rotatable about the pin 40 that extends in a direction perpendicular to the longitudinal axis direction A, and can be opened and closed toward the distal side A1. The first forceps piece 41 is connected to the first link 341 on the proximal end side A2 from the pin 40. The second forceps piece 42 is connected to the second link 342 on the proximal end side A2 from the pin 40. As the rod 33 advances, the first link 341 and the second link 342 move away from each other in the radial direction R, opening the first forceps piece 41 and the second forceps piece 42. As the rod 33 retracts, the first link 341 and the second link 342 approach each other in the radial direction R, closing the first forceps piece 41 and the second forceps piece 42.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

### EXAMPLE

A handle assembly, comprising:
a handle body;
a transmission member configured to transmit a high-frequency current;
a conductive connector extending in a direction intersecting a longitudinal axis of the transmission member; and
a conductive elastic body configured to bias the transmission member in a direction of extension of the connector and to press the transmission member against the connector,
wherein the elastic body contacts the transmission member in a state in which the elastic body is compressed, and
the transmission member is slidable relative to the connector in a state in which the transmission member is pressed against the connector.

What is claimed is:

1. A handle assembly, comprising:
a handle body;
a transmission member configured to transmit a high-frequency current;
a connector being conductive and extending in a direction intersecting a longitudinal axis of the transmission member; and
an elastic body,
wherein the elastic body contacts the transmission member, and
the transmission member is slidable relative to the connector in a state in which the transmission member is pressed against the connector.

2. The handle assembly according to claim 1, wherein
a first end of the connector has a plug capable of connecting a power cable that transmits the high-frequency current,
a second end of the connector has an insertion passage formed therein that extends in a longitudinal axis direction of the handle body,
the transmission member is inserted into the insertion passage, and
the transmission member is pressed against an inner surface of the insertion passage by the elastic body.

3. The handle assembly according to claim 1, wherein
the transmission member includes a pipe through which a wire passes, and
the elastic body directly contacts the pipe and presses the pipe against the connector.

4. The handle assembly according to claim 1, wherein the elastic body is a coiled compression spring.

5. The handle assembly according to claim 1, wherein the elastic body is sandwiched between the transmission member and the connector in a state in which the elastic body is compressed.

6. The handle assembly according to claim 1, wherein
the connector has a first end having a plug capable of connecting a power cable that transmits the high frequency current, and a second end against which the transmission member is pressed,
the connector has a flange located between the plug and the transmission member,
the elastic body is sandwiched between the transmission member and the flange in a state in which the elastic body is compressed, and
the elastic body urges the transmission member toward the second end.

7. The handle assembly according to claim 6, wherein
the elastic body is a compression spring; and
at least a part of the connector is inserted through the compression spring.

8. The handle assembly according to claim 2, wherein in a state in which the transmission member is pressed against the second end of the connector by the elastic body, the transmission member is deflected toward the second end of the connector.

9. The handle assembly according to claim 1, wherein in a state in which the transmission member is pressed against the connector, the transmission member is slidable relative to the connector in a direction around the longitudinal axis of the transmission member.

10. The handle assembly according to claim 1, wherein in a state in which the transmission member is pressed against the connector, the transmission member is slidable relative to the connector in a direction along a longitudinal axis of the transmission member.

11. A handle assembly, comprising:
a handle body;
a transmission member configured to transmit a high-frequency current;
a connector being conductive and extending in a direction intersecting the transmission member; and
a spring disposed in a compressed state between the transmission member and the connector,
wherein when the transmission member and the spring contact with each other, the transmission member, the spring, and the connector are electrically connected.

12. An endoscope treatment tool, comprising:
a sheath;
an end effector disposed at a distal end of the sheath;
a handle connected to a proximal end of the sheath;
a long member connected to the end effector; and
a transmission member configured to transmit high frequency current to the long member,
wherein the handle includes
a connector being conductive and extending in a direction intersecting the transmission member, and
an elastic body configured to press the transmission member against the connector,
wherein the transmission member is slidable relative to the connector in a state in which the transmission is pressed against the connector.

13. The endoscope treatment tool according to claim 12, wherein
a first end of the connector has a plug capable of connecting a power cable that transmits the high frequency current,
a second end of the connector has an insertion passage extending in a longitudinal axis direction of the handle,
the connector has a flange located between the plug and the transmission member,
the elastic body is sandwiched between the transmission member and the flange in a state in which the elastic body is compressed, and
the elastic body biases the transmission member toward the second end.

14. The endoscope treatment tool according to claim 12, wherein the long member is inserted through a sheath, and the end effector rotates relative to the sheath.

15. The endoscope treatment tool according to claim 12, wherein the long member has a wire.

16. The endoscope treatment tool according to claim 15, wherein
the transmission member has a pipe, and
the elastic body applies a biasing force to the pipe by contacting the pipe in a state in which the elastic body is compressed.

17. The endoscope treatment tool according to claim 12, wherein
the connector has an insertion passage through which the transmission member passes, and
the elastic body applies a biasing force to electrically connect the transmission member to at least a part of the insertion passage.

18. The endoscope treatment tool according to claim 12, wherein
the elastic body is a coil spring, and
a pitch of seat coils at both ends of the coil spring is narrower than that of other parts.

19. The endoscope treatment tool according to claim 12, wherein
the connector is disposed at a position spaced apart from the transmission member, and
the elastic body applies a biasing force to the transmission member to electrically connect the transmission member to the connector.

20. The endoscope treatment tool of claim 12, wherein the elastic bodies are arranged on both sides of the transmission member.
